(19) United States
(12) Patent Application Publication
(10) Pub. No.: US 2019/0079086 A1
(43) Pub. Date: Mar. 14, 2019

(54) DETECTION STRUCTURE AND PRODUCTION METHOD THEREOF

(71) Applicant: National Tsing Hua University, Hsinchu City (TW)

(72) Inventors: Sheng-Kai Wang, Taipei City (TW); Tse-Hsueh Lin, Taichung City (TW)

(73) Assignee: National Tsing Hua University, Hsinchu City (TW)

(21) Appl. No.: 16/024,928

(22) Filed: Jul. 2, 2018

(30) Foreign Application Priority Data
Sep. 14, 2017 (TW) 106131620

(51) Int. Cl. G01N 33/543; G01N 33/531

(52) U.S. Cl. CPC G01N 33/54393; G01N 33/531

(57) ABSTRACT

A detection structure including a substrate, a coating structure and a plurality of polyproline helix structure is provided. The substrate has a plurality of detecting regions. The coating structure is located on the substrate. The plurality of polyproline helix structure is located in each of the detecting region and on the coating structure. Each of the polyproline helix structures is composed of a plurality of proline monomers aligned in the first direction, aligned in the second direction and aligned in the third direction. The proline monomer aligned in the first direction is connected in the coating structure through a connecting structure, and the proline monomer aligned in the second direction is connected to at least two ligands. The two ligands on each of the polyproline helix structure has a fixed distance that can be adjusted. A production method of the detection structure above is also described.

### CROSS-REFERENCE TO RELATED APPLICATION

This application claims the priority benefit of Taiwan application serial no. 106131620, filed on Sep. 14, 2017. The entirety of the above-mentioned patent application is hereby incorporated by reference herein and made a part of this specification.

### BACKGROUND OF THE INVENTION

### 1. Field of the Invention

The present invention generally relates to a detection structure, in particular, relates to a detection structure for detecting multivalent interactions between ligands and proteins, and a production method thereof.

### 2. Description of Related Art

Multivalent interactions between proteins and ligands are essential in many biological processes including cell recognition and signal transduction etc. Knowing the spatial arrangement of the binding events between the ligands and proteins will greatly benefit the understanding and manipulation of such biological processes. Current investigations on multivalent interactions and spatial arrangement usually rely on protein structural studies and synthetic multivalent scaffolds. For example, some researchers have proposed to use DNA nanogrids to achieve a fixed distance between ligands, or proposed to use polymer stents for connecting ligands to study the interactions between ligands and proteins.

However, in using nanogrid technology, the spacing between ligands are too large, hence the investigations on multivalent interactions with protein is not ideal. Furthermore, there are also many problems with the use of polymer stents for detecting multivalent interactions with protein. Since the polymers do not have a fixed structure and may have a dispersed molecular weight, therefore, the ligands modified on the polymers cannot be easily controlled. In addition, the ligands connected on different polymer chains are also susceptible to cross-linking reactions during detection, hence, the multivalent interactions with proteins cannot be effectively and accurately detected. Accordingly, how to more effectively detect the multivalent interactions between ligands and proteins, and how to effectively regulate and control the distance between the ligands is a topic that is being actively researched.

### SUMMARY OF THE INVENTION

Accordingly, the present invention provides a detection structure and a production method thereof, wherein the multivalent interactions between the ligands and proteins can be more effectively detected.

The detection structure of the present invention includes a substrate, a coating structure and a plurality of polyproline helix structures. The substrate has a plurality of detecting regions. The coating structure is located on the substrate. The plurality of polyproline helix structures is respectively located in each of the detecting regions and on the coating structure. Each of the polyproline helix structures is composed of a repeating helical arrangement of a plurality of proline monomers aligned in a first direction, a plurality of proline monomers aligned in a second direction, and a plurality of proline monomers aligned in a third direction. At least one of the plurality of proline monomers aligned in the first direction is connected to the coating structure through a connecting structure, or a N-terminal or C-terminal of the plurality of polyproline helix structures is connected to the substrate through covalent bonds. The plurality of proline monomers aligned in the second direction is connected to at least two ligands, wherein the two ligands on each of the polyproline helix structures has a fixed distance that can be adjusted. The fixed distance is a spacing that is generated by the secondary structure of the polyproline.

In an embodiment of the invention, the coating structure is a fluorous coating structure, and the connecting structure contains perfluorinated alkyl moiety, and the plurality of proline monomers aligned in the first direction is connected to the coating structure by a non-covalent interaction between the perfluorinated alkyl moiety and the fluorous coating structure.

In an embodiment of the invention, the perfluorinated alkyl moiety is a perfluorinated alkyl moiety having more than three carbons.

In an embodiment of the invention, each of the polyproline helix structures contain at least two chains of the perfluorinated alkyl moiety connected to the coating structure.

In an embodiment of the invention, all the proline monomers aligned in the first direction is not adjacent to one another, all the proline monomers aligned in the second direction is not adjacent to one another, and all the proline monomers aligned in the third direction is not adjacent to one another.

In an embodiment of the invention, the fixed distance is 0.9±0.1 nm, 1.8±0.1 run, 2.7±0.1 nm, 3.6 nm±0.1 nm or 4.5 nm±0.1 nm.

In an embodiment of the invention, the plurality of proline monomers aligned in the second direction is connected to the at least two ligands through covalent bonding.

In an embodiment of the invention, the detection structure further comprises a plurality of dummy structures, respectively located in each of the detecting regions and on the coating structure, wherein the plurality of dummy structures is connected to the coating structure by a non-covalent interaction through perfluorinated alkyl moiety.

In an embodiment of the invention, a ratio between the plurality of dummy structures and the plurality of polyproline helix structures in each of the detecting regions is 3:1 or greater than 3:1.

A production method of a detection structure of the present invention includes the following steps. A substrate is provided, the substrate has a plurality of detecting regions. A coating structure is coated on the substrate. A plurality of polyproline helix structures is provided, wherein each of the polyproline helix structures is composed of a repeating helical arrangement of a plurality of proline monomers aligned in a first direction, a plurality of proline monomers aligned in a second direction, and a plurality of proline monomers aligned in a third direction. The plurality of proline monomers aligned in the first direction is modified with at least one connecting structure, so that the plurality of proline monomers aligned in the first direction is connected to the coating structure in each of the detecting regions through the connecting structure. The plurality of proline monomers aligned in the second direction is connected with at least two ligands, so that the two ligands on each of the polyproline helix structures has a fixed distance that can be adjusted. A plurality of dummy structures is provided, the plurality of dummy structures is connected to the coating structure in each of the detecting regions, so that the plurality of dummy structures and the plurality of polyproline helix structures are adjacent to one another.

In an embodiment of the invention, the coating structure is a fluorous coating structure, and the plurality of proline monomers aligned in the first direction is modified with perfluorinated alkyl moiety of the connecting structure, so that the plurality of proline monomers aligned in the first direction is connected to the coating structure in each of the detecting regions through the perfluorinated alkyl moiety.

In an embodiment of the invention, the plurality of proline monomers aligned in the first direction is modified with perfluorinated alkyl moiety of the connecting structure, and the perfluorinated alkyl moiety is a perfluorinated alkyl moiety having more than three carbons.

In an embodiment of the invention, the plurality of proline monomers aligned in the first direction is modified with at least two chains of the perfluorinated alkyl moiety of the connecting structure.

In an embodiment of the invention, the plurality of proline monomers aligned in the second direction is connected to the at least two ligands through covalent bonding.

In an embodiment of the invention, the plurality of dummy structures is connected to the coating structure by a non-covalent interaction through perfluorinated alkyl moiety.

In an embodiment of the invention, a ratio between the plurality of dummy structures and the plurality of polyproline helix structures in each of the detecting regions is 3:1 or greater than 3:1.

In an embodiment of the invention, the fixed distance is 0.9±0.1 nm, 1.8±0.1 nm, 2.7±0.1 nm, 3.6 nm±0.1 nm or 4.5 nm±0.1 nm.

Based on the above, in the detection structure and the production method of the present invention, the plurality of polyproline helix structures is utilized for connection within the coating structure of the substrate. In addition, the two ligands connected on the polyproline helix structure has a fixed distance that can be adjusted. Therefore, the detection structure of the present invention can more effectively detect the multivalent interactions between the ligands and proteins. Further, the distance between the ligands can be confirmed and adjusted to achieve better detection results.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a schematic diagram of a detection structure in accordance with an embodiment of the invention.

FIG. 1B is a sectional view of a plurality of detecting regions in the substrate according to FIG. 1A.

FIG. 2A is a front view molecular structure diagram of a polyproline helix structure in accordance with an embodiment of the invention.

FIG. 2B is a side view molecular structure diagram of a polyproline helix structure in accordance with an embodiment of the invention.

FIG. 3A is a side view diagram of a polyproline helix structure after modification in accordance with an embodiment of the invention.

FIG. 3B is a side view diagram of a polyproline helix structure after modification in accordance with another embodiment of the invention.

FIG. 4 is a process flow diagram for modifying perfluorinated alkyl moiety on the proline monomer in accordance with an embodiment of the invention.

FIG. 5 is an experimental result diagram of testing the binding strengths of perfluorinated alkyl moiety in accordance with an embodiment of the invention.

FIG. 6A is a process flow diagram for bonding a polyproline helix structure with two ligands in accordance with an embodiment of the invention.

FIG. 6B is a process flow diagram for bonding a polyproline helix structure with two ligands in accordance with another embodiment of the invention.

FIG. 7A is a schematic top view diagram of a detecting region in a comparative example of the present invention.

FIG. 7B is a schematic top view diagram of a detecting region in an embodiment of the present invention.

FIG. 8A is a surface dissociation constant evaluation diagram of the polyproline helix structure at different ratios in accordance with an embodiment of the invention.

FIG. 8B is a surface dissociation constant evaluation diagram of the polyproline helix structure at different ratios in accordance with another embodiment of the invention.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

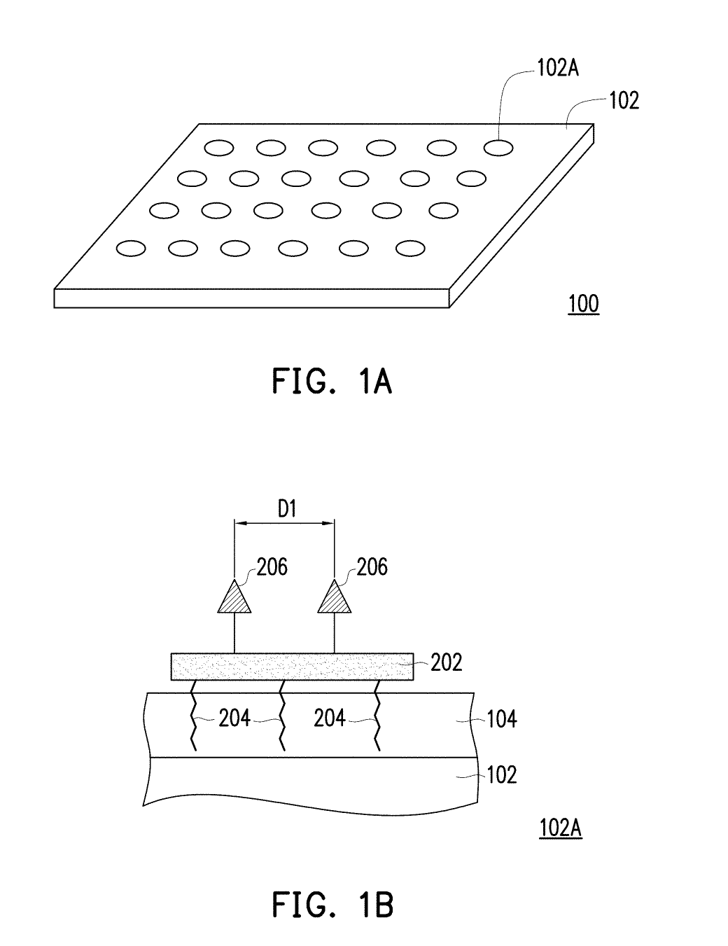

FIG. 1A is a schematic diagram of a detection structure in accordance with an embodiment of the invention. FIG. 1B is a sectional view of a plurality of detecting regions in the substrate according to FIG. 1A. Referring to both FIG. 1A and FIG. 1B, the detection structure 100 of the present invention includes a substrate 102, wherein the substrate 102 has a plurality of detecting regions 102A. The substrate 102 is, for example, a glass carrier substrate, but not limited thereto. For example, a material of the substrate 102 may be any suitable substrate material commonly used for detection and analysis. The detecting regions 102A are, for example, a region in the detection structure 100 that can be used to detect the multivalent interactions between ligands and proteins. In an embodiment of the invention, the detection structure 100 may be microarray chips, but not limited thereto. In other embodiments, the detection structure 100 may be any detection device that includes detecting regions 102A. For example, the detection structure 100 may be surface plasmon resonance (SPR) or quartz crystal microbalance (QCM) detection devices. However, the disclosure is not limited thereto. In some other embodiments, only one detecting region 102A is included over the surface of the substrate 102. For example, slides may be used as the substrate 102, wherein the entire surface of the substrate 102 may include only one detecting region 102A. It is noted that the arrangement of the detecting region 102A may be adjusted based on requirement.

In the present embodiment, the coating structure 104 is located on the substrate 102, and a plurality of polyproline helix structures 202 is respectively located in each of the detecting regions 102A and on the coating structure 104. The coating structure 104 is, for example, a structure that is suitable to be connected to the polyproline helix structures 202. For example, the polyproline helix structures 202 are connected to the coating structure 104 through a connecting structure 204. In the present embodiment, the coating structure 104 used is, for example, a fluorous coating structure, and the connecting structure 204, for example, contains perfluorinated alkyl moiety. In the present embodiment, each of the polyproline helix structures 202 contain at least two chains of the perfluorinated alkyl moiety connected to the coating structure 104. More specifically, the number of perfluorinated alkyl moiety can be two chains, three chains or more, as long as it is possible to stably connect the perfluorinated alkyl moiety to the coating structure 104. Accordingly, proline monomers in the polyproline helix structures 202 may be connected to the coating structure 104 by a non-covalent interaction between the perfluorinated alkyl moiety and the fluorous coating structure. In some specific embodiments, the coating structure 104 may also be omitted as long as the polyproline helix structures 202 are connected onto the substrate 102. For example, in another embodiment, a N-terminal or C-terminal of the polyproline helix structures 202 are connected onto the substrate 102 through covalent bonding reactions.

Furthermore, referring to FIG. 1B, the polyproline helix structures 202 are connected to at least two ligands 206, wherein the two ligands 206 on each of the polyproline helix structures 202 have a fixed distance D1 that can be adjusted. The fixed distance D1 is, for example, a spacing that is generated by the secondary structure of the polyproline. In the present embodiment, the fixed distance that may be adjusted intends to mean that the distance between the two ligands 206 on each of the polyproline helix structures 202 are kept the same/fixed, however, the range of this distance may be adjusted according to requirement. In the present embodiment, the fixed distance D1 is approximately 0.9±0.1 nm, 1.8±0.1 nm, 2.7±0.1 nm, 3.6 nm±0.1 nm or 4.5 nm±0.1 nm. However, the distance between the two ligands 206 on each of the polyproline helix structures 202 are kept the same. Although the fixed distance D1 of the present embodiment is based on the examples of 0.9±0.1 nm, 1.8±0.1 nm, 2.7±0.1 nm, 3.6 nm±0.1 nm or 4.5 nm±0.1 nm, however, the invention is not limited thereto. In other embodiments, the fixed distance D1 may be altered or adjusted depending on the multivalent interaction to be detected.

Next, the molecular structure of the polyproline helix structures 202 and how the polyproline helix structures 202 are connected to the coating structure 104 and attached with the two ligands 206 will be described in detail.

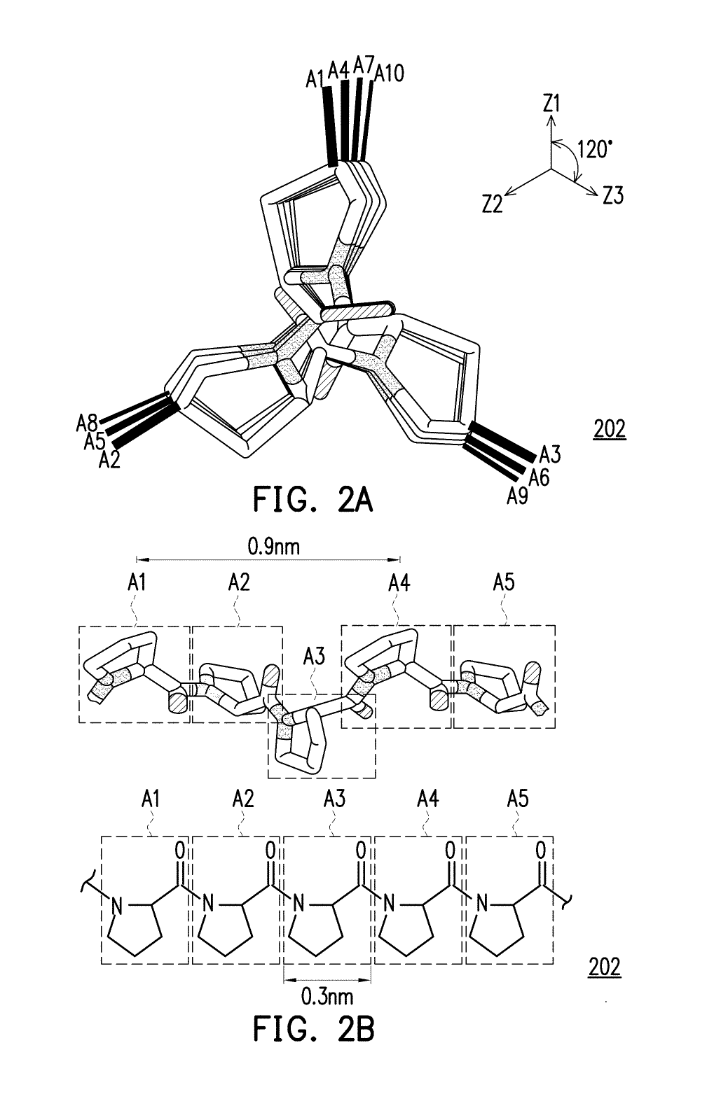

FIG. 2A is a front view molecular structure diagram of a polyproline helix structure in accordance with an embodiment of the invention. FIG. 2B is a side view molecular structure diagram of a polyproline helix structure in accordance with an embodiment of the invention. Referring to both FIG. 2A and FIG. 2B, the polyproline helix structure 202 is composed of a repeating helical arrangement of a plurality of proline monomers substantially aligned in a first direction Z1, a plurality of proline monomers substantially aligned in a second direction Z2, and a plurality of proline monomers substantially aligned in a third direction Z3. More specifically, the polyproline helix structure 202 used in the present invention is, for example, a polyproline type II (PPII) helix structure.

As shown in, FIG. 2A, the first proline monomer A1 in the polyproline helix structure 202 is substantially aligned in the first direction Z1, the second proline monomer A2 is substantially aligned in the second direction Z2, and the third polyproline monomer A3 is substantially aligned in the third direction Z3. Subsequently, the fourth polyproline monomer A4 will return to be substantially aligned in the first direction Z1. Herein, counting from the first proline monomer A1 until a proline monomer returns to approximately the same direction, this distance in the polyproline helix structures 202 may be referred as a “1 turn” structure. Similarly, the subsequent fifth proline monomer A5, sixth proline monomer A6, seventh proline monomer A7, eighth proline monomer A8, ninth proline monomer A9 and the tenth proline monomer A10 will have a repeating arrangement that is sequentially aligned in the second direction Z2, aligned in the third direction Z3, and aligned back to the first direction Z1 to form a helical structure. In FIG. 2A, since the polyproline helix structure 202 has ten proline monomers in total, therefore, this may be viewed as a “3 turns” structure. In addition, as shown in FIG. 2A, the first direction Z1, the second direction Z2 and the third direction Z3 are respectively separated by approximately 120 degrees.

Furthermore, as shown in FIG. 2B, each of the proline monomers and the next proline monomer aligned in the same direction are spaced apart by a distance of approximately 0.9±0.1 nm. Referring to FIG. 2B, each of the proline monomers have a length of approximately 0.3 nm, and every three proline monomers is counted as 1 turn. Therefore, the distance between the first proline monomer A1 and the fourth proline monomer A4 is approximately 0.9±0.1 nm, and the distance between the second proline monomer A2 and the fifth proline monomer A5 is also approximately 0.9±0.1 nm, and so forth. In addition, as can be noted in FIG. 2A and FIG. 2B, the proline monomers (A1, A4, A7, A10) aligned in the first direction Z1 are not adjacent to one another, the proline monomers (A2, A5, A8) aligned in the second direction Z2 are not adjacent to one another, and the proline monomers (A3, A6, A9) aligned in the third direction are not adjacent to one another. In the embodiments of the present invention, the proline monomers aligned in the same direction (first direction Z1, second direction Z2 or third direction Z3) will partially undergo the same modification to achieve the detection structure 100 having ligands 102 with adjustable distances.

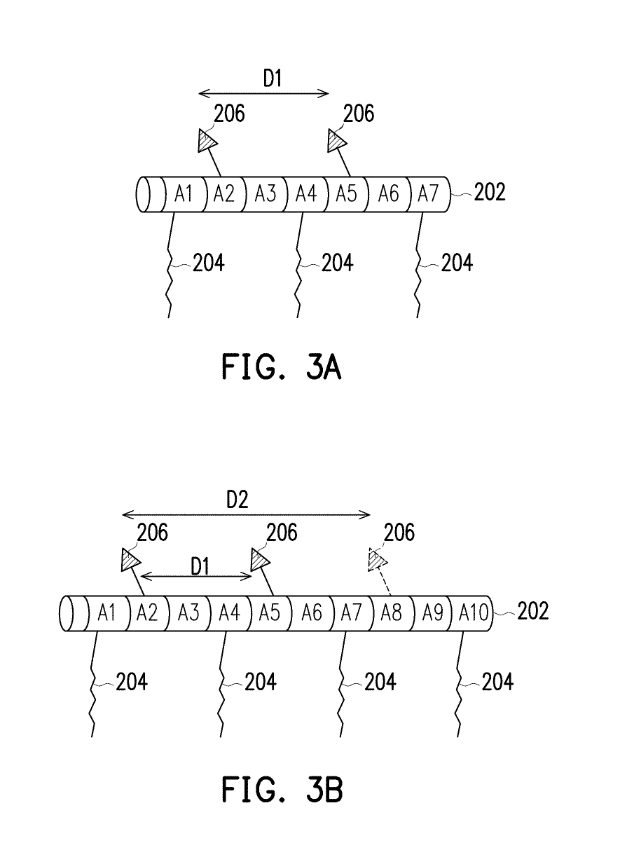

FIG. 3A is a side view diagram of a polyproline helix structure after modification in accordance with an embodiment of the invention. Referring to FIG. 3A, the polyproline helix structure 202 of the invention is preferably a polyproline helix structure 202 having more than 2 turns (with a total of seven proline monomers). As shown in FIG. 3A, in the present embodiment, the proline monomers (first proline monomer A1, fourth proline monomer A4, seventh proline monomer A7) aligned in the first direction Z1 is connected to the coating structure 104 shown in FIG. 1B through at least one connecting structure 204. In the present embodiment, the polyproline helix structure 202 contains three chains of perfluorinated alkyl moiety as the connecting structure 204, so that it can be connected to the coating structure 104, which is a fluorous coating structure. However, it should be noted that the number of perfluorinated alkyl moiety is not limited to three chains, but can be two chains, four chains or more. In the case of a polyproline helix structure 202 having 2 turns, the three chains of perfluorinated alkyl moiety are respectively located on the first proline monomer A1, the fourth proline monomer A4, and the seventh proline monomer A7, so as to obtain a structure that is stably connected to the coating structure 104. However, the present invention is not limited thereto. In other embodiments, it is also possible to use only two chains of the connecting structure 204 respectively disposed on the first proline monomer A1 and the seventh proline monomer A7 to provide connection within the coating structure 104. Additionally, more than three chains of perfluorinated alkyl moiety may be used for connection to the coating structure 104. Furthermore, in the present embodiment, the proline monomers aligned in the second direction Z2, for example, the second proline monomer A2 and the fifth proline monomer A5 is connected to at least two ligands 206, wherein the two ligands 206 on each of the polyproline helix structures 202 has a fixed distance D1 that can be adjusted. In the embodiment of FIG. 3A, the fixed distance D1 is 0.9±0.1 nm, which equals to the distance of 1 turn. It should be noted that depending on the length of the polyproline helix structures 202, the fixed distance between the two ligands 206 may be different, and the number of connecting structure 204 used may also be different.

FIG. 3B is a side view diagram of a polyproline helix structure after modification in accordance with another embodiment of the invention. The polyproline helix structure 202 shown in FIG. 3B is similar to the polyproline helix structure 202 shown in FIG. 3A, the difference being that the polyproline helix structures 202 have different lengths. Referring to FIG. 3B, the polyproline helix structure 202 is a polyproline helix structure 202 having 3 turns (with a total of ten proline monomers). In the case of a polyproline helix structure 202 having 3 turns (with a total of ten proline monomers), the connecting structure 204 may, for example, include four chains of perfluorinated alkyl moiety respectively located on the first proline monomer A1, the fourth proline monomer A4, the seventh proline monomer A7 and the tenth proline monomer A10. However, the present invention is not limited thereto. In other embodiments, the number of perfluorinated alkyl chains may be adjusted based on requirement. In addition, the proline monomers aligned in the second direction Z2, for example, the second proline monomer A2, the fifth proline monomer A5 or the eighth proline monomer A8 may be selectively disposed with at least two ligands 206. For instance, the two ligands 206 may be disposed on the second proline monomer A2 and the fifth proline monomer A5 so as to achieve a fixed distance D1 of 0.9±0.1 nm. Alternatively, in another embodiment, the two ligands 206 may be disposed on the second proline monomer A2 and the eighth proline monomer A8 so as to achieve a fixed distance D2 of 1.8±0.1 nm. That is to say, the distance between the two ligands 206 can be adjusted depending on the protein and multivalent interaction to be detected, however, the distance between the two ligands 206 on each of the polyproline helix structures 202 are still kept the same.

In FIG. 3A and FIG. 3B, although the polyproline helix structures 202 having 2 turns or 3 turns are taken as examples, however, it should be noted that the length of the polyproline helix structures 202 may be adjusted based on requirement. Moreover, the distance between the two ligands 206 may also be adjusted depending on the length of the polyproline helix structures 202. For example, in other embodiments, when the polyproline helix structure 202 have 4 turns, then the distance between the two ligands 206 can reach a maximum of 2.7±0.1 nm (distance of 3 turns), and have a minimum of 0.9±0.1 nm (distance of 1 turn). Besides, in the present embodiment, when the connecting structure 204 contains perfluorinated alkyl moiety, then the length of the perfluorinated alkyl moiety is not particularly limited, and may for example be perfluorinated alkyl moiety having more than three carbons. In specific embodiments, the perfluorinated alkyl moiety is, for example, a C₃F₇ perfluorinated alkyl group, a C₅F₁₁ perfluorinated alkyl group or a C₇F₅ perfluorinated alkyl group. In addition, the perfluorinated alkyl moiety should in fact include other embodiments where the perfluorinated alkyl moiety are elongated with other atoms or functional groups, and should not be limited to the perfluorinated alkyl moiety described above. The method of modifying the proline monomers with perfluorinated alkyl moiety will be described below.

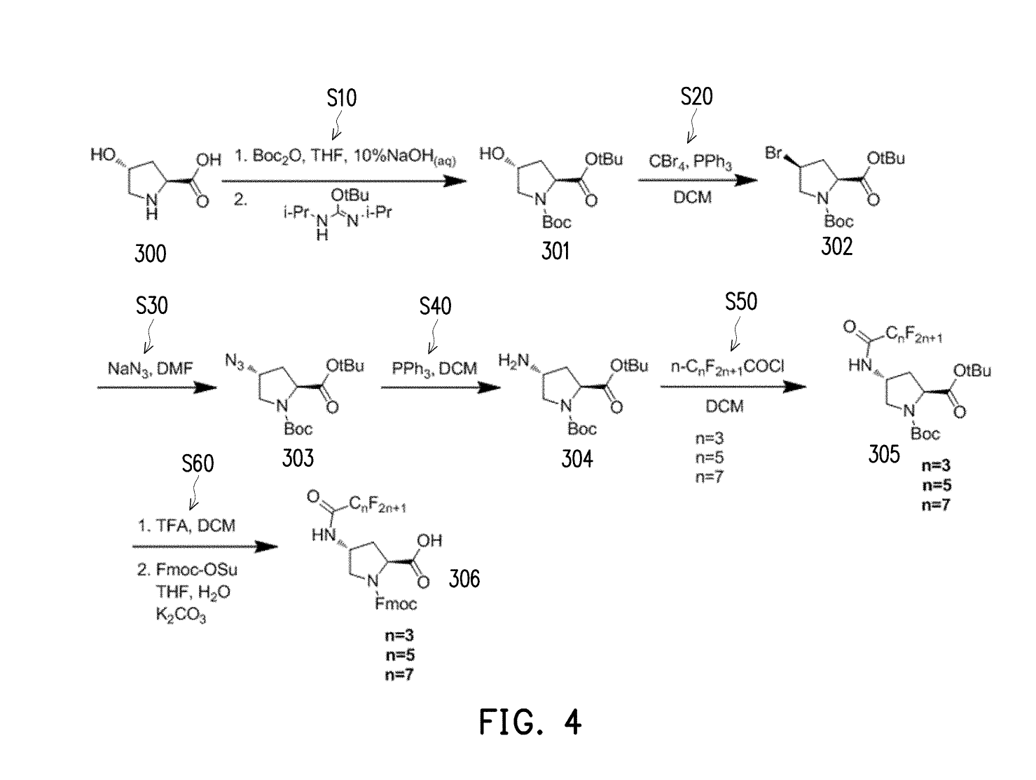

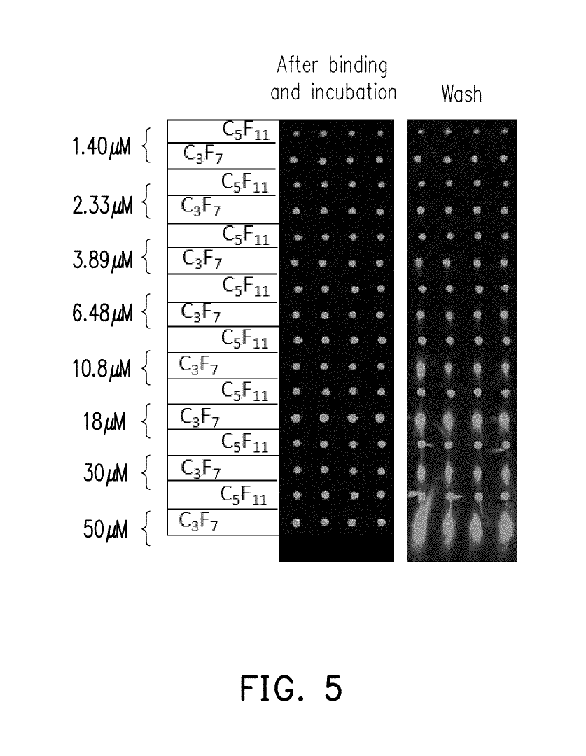

FIG. 4 is a process flow diagram for modifying perfluorinated alkyl moiety on the proline monomer in accordance with an embodiment of the invention. Referring to FIG. 4, a compound 300 (4-hydroxy-L-proline) is first provided. In step S10, a tert-butoxycarbonyl (Boc) protecting group is introduced to the amino group in compound 300 in a first step, and a t-butyl ester protecting group is introduced to a position of the acid (COOH) in a second step so as to obtain compound 301. Next, step S20 is performed by adding CBr₄, PPh₃ and DCM for reaction, so that the hydroxyl group of compound 301 is replaced with a bromo group to form compound 302. Next, NaN₃ and DMF are added in step S30 for reaction, so that the bromo group of compound 302 is replaced with an azide to form compound 303. Subsequently, in step S40, the azide may be converted to an amine by using PPh₃ and DCM for reaction so as to form compound 304. In step S50, perfluorinated alkyl moiety having different lengths (n-CₙF₂ₙ₊₁COCl; n=3, 5, 7) is added in a DCM solvent for reaction with compound 304, and the perfluorinated alkyl moiety may be connected to the proline monomer so as to form compound 305. At last, in step S60, trifluoroacetic acid is used in a first step to remove the protecting groups, and a fluorenylmethyloxycarbonyl (Fmoc) protecting group may then be introduced to the amine in a second step to obtain compound 306. Accordingly, the compound 306 may be used as a building block for solid-phase peptide synthesis (SPPS) so as to form the desired polyproline helix structures 202.

As described previously, in the embodiments of the present invention, the perfluorinated alkyl moiety connected on the polyproline helix structures 202 may for example, be C₃F₇ perfluorinated alkyl groups, C₅F₁₁ perfluorinated alkyl groups, or C₇F₁₅ perfluorinated alkyl groups. In other embodiments, perfluorinated alkyl moiety having different lengths may also be formed by referring to the steps shown in FIG. 4. In order to confirm the binding strengths of perfluorinated alkyl moiety of different lengths to the fluorous coating structure (coating structure 104), the polyproline helix structures 202 having different perfluorinated alkyl lengths are labelled with fluorescent tags and tested. FIG. 5 is an experimental result diagram of testing the binding strengths of perfluorinated alkyl moiety in accordance with an embodiment of the invention. Referring to FIG. 5, C₃F₇ perfluorinated alkyl group and C₅F₁₁ perfluorinated alkyl group are tested in the current embodiment, and the tested concentrations are in a range of 1.40 μM to 50 μM. In the two columns of fluorescent diagrams, the diagram on the left shows the fluorescent print of the polyproline helix structures 202 after binding and incubation for a certain period of time, and the diagram on the right shows the fluorescent intensity detected after the polyproline helix structures 202 binds to the fluorous coating structure and being washed with PBS. The experimental results revealed that the C₃F₇ perfluorinated alkyl group was partly washed away at higher concentrations (starting from about 10.8 μM). In comparison, the C₅F₁₁ perfluorinated alkyl group retained its original fluorescent intensity even at the highest concentration after being washed with PBS. Based on the experimental results above, it can be noted that the C₃F₇ perfluorinated alkyl group has lower binding strengths with the fluorous coating structure (coating structure 104), hence, may be easily washed off by PBS. In comparison, the C₅F₁₁ perfluorinated alkyl group has stronger binding strengths and stability with the fluorous coating structure (coating structure 104), hence, it is the more preferred chain length.

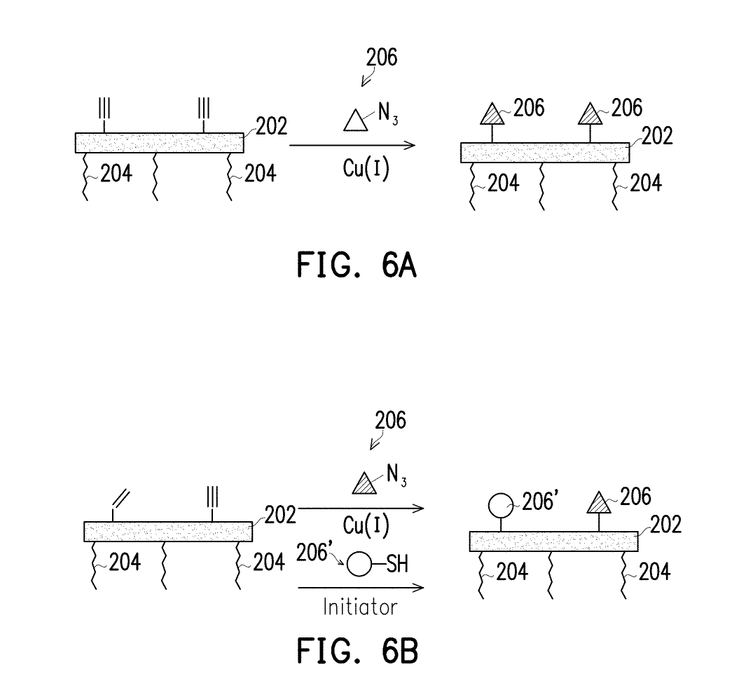

Next, the method of attaching the ligands 206 onto the polyproline helix structures 202 will be described. FIG. 6A is a process flow diagram for bonding a polyproline helix with two ligands in accordance with an embodiment of the invention. Referring to FIG. 6A, in some embodiments, if the polyproline helix structures 202 has a 2 turn (with seven proline monomers in total) structure, then the proline monomers (A2, A5) aligned in the second direction Z2 are modified with at least two Alkynes. For example, 4-propargyloxy proline may be used as a building block for solid-phase peptide synthesis (SPPS) to form a polyproline helix structure 202 having alkynes. As shown in FIG. 6A, the at least two alkynes on the polyproline helix structure 202 may be reacted with azides (N₃) on at least two ligands 206, so that the proline monomers (A2, A5) aligned in the second direction Z2 are bonded to the at least two ligands 206. More specifically, the polyproline helix structure 202 is attached with ligands 206 through a Cu(I) catalyzed alkyne-azide cycloaddition reaction (CuAAC).

FIG. 6B is a process flow diagram for bonding a polyproline helix with two ligands in accordance with another embodiment of the invention. As shown in FIG. 6A, the polyproline helix structure 202 is attached with two of the same ligands 206, however, the present invention is not limited thereto. In the embodiment of FIG. 6B, the polyproline helix structure 202 is attached with two different ligands 206 and 206′. In the embodiment of FIG. 6B, if the polyproline helix structure 202 has a 2 turn (with seven proline monomers in total) structure, then the proline monomers (A2, A5) aligned in the second direction Z2 are modified with an alkyne and an alkenyl group. The method of reacting the alkyne with the azide (N₃) on the ligand 206 is the same as shown in the embodiment of FIG. 6A. Furthermore, in the present embodiment, the alkenyl group on the polyproline helix structure 202 can be further reacted with a thiol group (SH) on the ligand 206′, so that the proline monomers (A2, A5) aligned in the second direction Z2 are bonded to the at least two ligands 206 and 206′. More specifically, the polyproline helix structure 202 is attached with ligands 206′ through a thiol-ene reaction.

From the embodiments of FIG. 6A and FIG. 6B, it will be appreciated that the arrangement of ligands (206 or 206′) is not particularly limited, and this may be adjusted depending on the proteins and multivalent interactions to be detected. Furthermore, the distance between the two ligands (206 or 206′) may be adjusted based on the length of the polyproline helix structure 202 or actual requirements. In the embodiments of FIG. 6A and FIG. 6B, the polyproline helix structure 202 modified with alkyne or alkenyl groups are taken as an example, however, the present invention is not limited thereto. In other embodiments, the proline monomers (A2, A5 etc.) aligned in the second direction Z2 are bonded to at least two ligands (206/206′) through covalent bonding. That is, as long as the proline monomers (A2, A5 etc.) aligned in the second direction Z2 are covalently bonded to the ligands, this will be sufficient to achieve the invention.

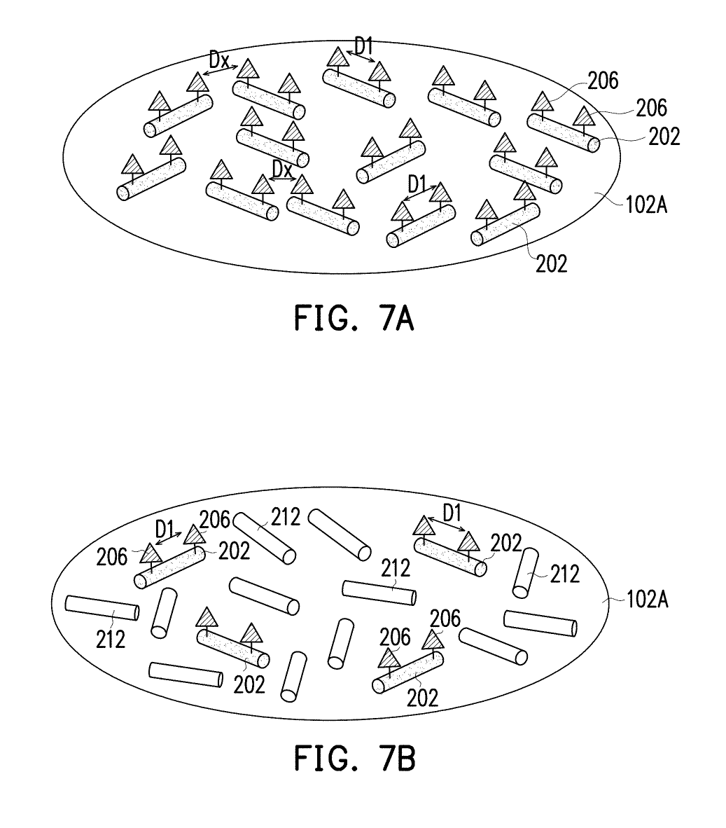

In the above embodiments, polyproline helix structure 202 connected to the coating structure 104 (FIG. 1B), and ligands (206 or 206′) attached on the polyproline helix structure 202 are being described. However, in the embodiments of the present invention, a plurality of dummy structures connected to the coating structure 104 may further be included. FIG. 7A is a schematic top view diagram of a detecting region in a comparative example of the present invention. As shown in FIG. 7A, the two ligands 206 on each of the polyproline helix structures 202 have a fixed distance D1. The fixed distance is used for detecting the multivalent interactions between the ligands and proteins. If the surface density of the polyproline helix structures 202 in the detecting region 102A is too high, then a ligand 206 on one of the polyproline helix structure 202 may form an unintended distance Dx with another ligand 206 on an adjacent polyproline helix structure 202. As the unintended distance Dx may correspond to binding of other proteins, thus if the surface density of the polyproline helix structure 202 is too high, then the multivalent interactions may not be efficiently and accurately detected. That is, there is a need to add a plurality of dummy structures to connect within the coating structure 104 so as to avoid the occurrence of the unintended distance Dx.

FIG. 7B is a schematic top view diagram of a detecting region in an embodiment of the present invention. As shown in FIG. 7B, the polyproline helix structure 202 and the dummy structure 212 may be connected to the coating structure 104 (FIG. 1B) in the detecting region 102A. In an embodiment of the present invention, the dummy structures 212 may, for example, be a polyproline helix structure without ligands 206 attached thereto, or may be other fluorine containing molecules or carriers. That is, since the ligands 206 are not provided, the dummy structures 212 does not actually bind to the proteins, and therefore will not affect the detection results. Similarly, the dummy structures 212 may be connected to the coating structure 104 (FIG. 1B) by a non-covalent interaction of the perfluorinated alkyl moiety. As shown in FIG. 7B, in the detecting region 102A, since the polyproline helix structures 202 are maintained at a specific ratio to the dummy structures 212, the surface density of the polyproline helix structures 202 will not become too high, thus the occurrence of the unintended distance Dx can be avoided. In addition, in some embodiments, in order to avoid the situation where the surface density of the polyproline helix structures 202 is too low and the multivalent interactions cannot be effectively detected, a ratio between the dummy structures 212 and the polyproline helix structures 202 in each of the detecting regions 102A is 3:1 or greater than 3:1. Herein, a range of the ideal ratio between the dummy structures 212 and the polyproline helix structures 202 is determined by applying a series of ratio changes. Furthermore, in other embodiments, it is also possible that the dummy structures 212 are not provided, but the density of the polyproline helix structures 202 needs to be spatially decreased by other means (for example, adjusting the surface density) to avoid the occurrence of the unintended distance Dx.

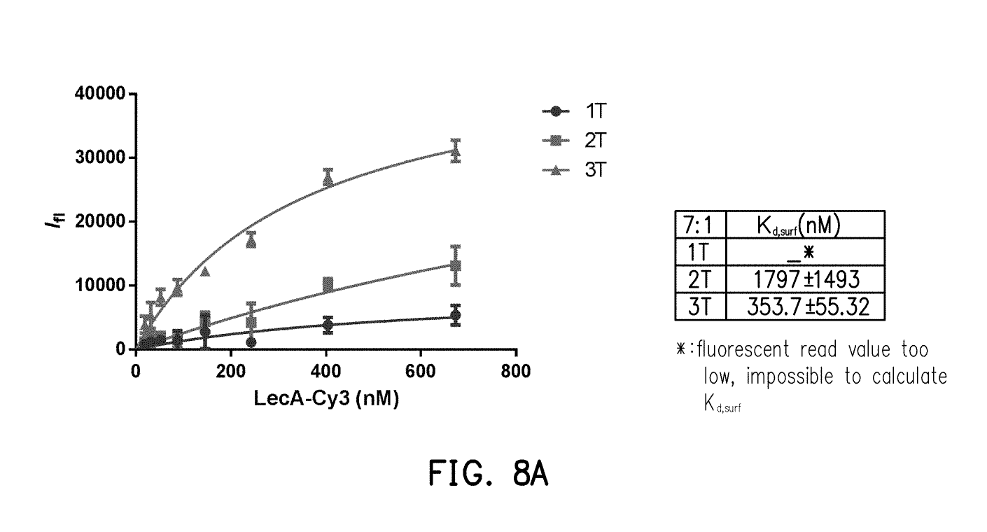

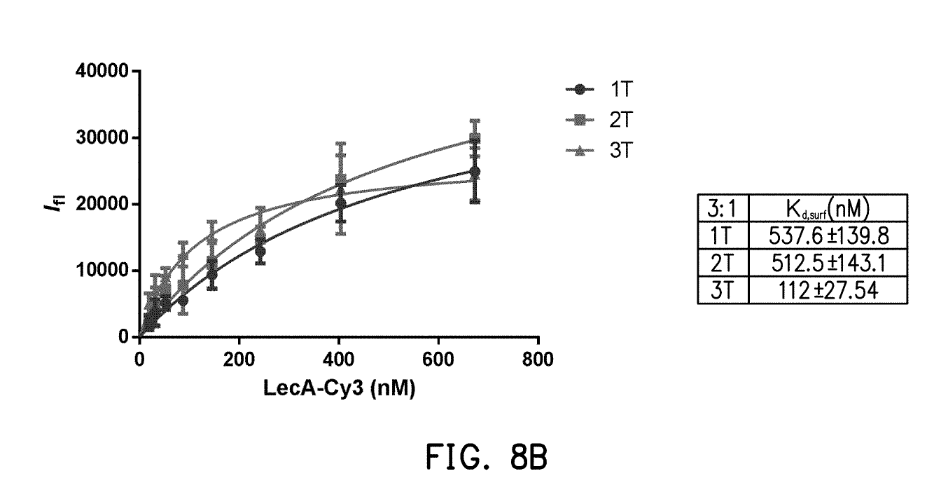

FIG. 8A is a surface dissociation constant evaluation diagram of the polyproline helix structure at different ratios in accordance with an embodiment of the invention. FIG. 8B is a surface dissociation constant evaluation diagram of the polyproline helix structure at different ratios in accordance with another embodiment of the invention. Referring to the embodiments of FIG. 8A and FIG. 8B, the ratio between the dummy structures 212 and the polyproline helix structures 202 is set to be 7:1 (FIG. 8A) or 3:1 (FIG. 8B). That is, in the embodiment of FIG. 8A, there are about 87.5% of the dummy structures 212 and about 12.5% of the polyproline helix structures 202 that exists. In the embodiment of FIG. 8B, there are about 75% of the dummy structures 212 and about 25% of the polyproline helix structures 202 that exists. Furthermore, in the embodiments of FIG. 8A and FIG. 8B, the two ligands 206 on the polyproline helix structures 202 may have different distances (1T: distance of 1 turn, 2T: distance of 2 turns, 3T: distance of 3 turns).

In the embodiments of FIG. 8A and FIG. 8B, the ligands 206 (galactose ligands) having different distances were mixed with different concentrations of protein (LecA-Cy3) at different ratios (3:1 or 7:1), and the surface dissociation constant (K_{d, surf}) was determined to evaluate the binding strengths of the ligands 206 with protein. Herein, a lower surface dissociation constant (K_{d, surf}) represents a better binding between the ligands 206 with the protein. As shown from the experimental results of FIG. 8A and FIG. 8B, it can be noted that the ligands 206 with a distance of 3 turns (3T: 2.7±0.1 nm) have the lowest surface dissociation constant. That is, the ligands 206 with a distance of 3 turns have the best binding with the LecA protein. In addition, from the experiments shown in FIG. 8A and FIG. 8B, when a ratio between the dummy structures 212 and the polyproline helix structures 202 is increased to above 3:1, this may help to reduce the cross-linking of proteins across different polyproline helix structures 202. That is, the unintended distance Dx may be avoided.

According to the above, in the detection structure and the production method of the present invention, the plurality of polyproline helix structures is utilized for connection within the coating structure of the substrate. In addition, the two ligands connected on the polyproline helix structure has a fixed distance that can be adjusted. Therefore, the detection structure of the present invention can more effectively detect the multivalent interactions between the ligands and proteins. Further, the distance between the ligands can be confirmed and adjusted to achieve better detection results. Moreover, dummy structures may be added into the detecting region so as to avoid the occurrence of unintended distances between the ligands, which may affect the detection results.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A detection structure, comprising:
a substrate, the substrate has a plurality of detecting regions;
a coating structure, located on the substrate; and
a plurality of polyproline helix structures, respectively located in each of the detecting regions and on the coating structure, wherein each of the polyproline helix structures is composed of a repeating helical arrangement of a plurality of proline monomers aligned in a first direction, a plurality of proline monomers aligned in a second direction, and a plurality of proline monomers aligned in a third direction,
at least one of the plurality of proline monomers aligned in the first direction is connected to the coating structure through a connecting structure or a N-terminal or C-terminal of the plurality of polyproline helix structures is connected to the substrate through covalent bonds, and
the plurality of proline monomers aligned in the second direction is connected to at least two ligands, wherein the two ligands on each of the polyproline helix structures has a fixed distance that can be adjusted.

2. The detection structure according to claim 1, wherein the coating structure is a fluorous coating structure, and the connecting structure contains perfluorinated alkyl moiety, and the plurality of proline monomers aligned in the first direction is connected to the coating structure by a non-covalent interaction between the perfluorinated alkyl moiety and the fluorous coating structure.

3. The detection structure according to claim 2, wherein the perfluorinated alkyl moiety is a perfluorinated alkyl moiety having more than three carbons.

4. The detection structure according to claim 2, wherein each of the polyproline helix structures contain at least two chains of the perfluorinated alkyl moiety connected to the coating structure.

5. The detection structure according to claim 1, wherein the plurality of proline monomers aligned in the first direction is not adjacent to one another, the plurality of proline monomers aligned in the second direction is not adjacent to one another, and the plurality of proline monomers aligned in the third direction is not adjacent to one another.

6. The detection structure according to claim 1, wherein the fixed distance is 0.9±0.1 nm, 1.8±0.1 nm, 2.7±0.1 nm, 3.6 nm±0.1 nm or 4.5 nm±0.1 nm.

7. The detection structure according to claim 1, wherein the plurality of proline monomers aligned in the second direction is connected to the at least two ligands through covalent bonding.

8. The detection structure according to claim 1, further comprising a plurality of dummy structures, respectively located in each of the detecting regions and on the coating structure, wherein the plurality of dummy structures is connected to the coating structure by a non-covalent interaction through perfluorinated alkyl moiety.

9. The detection structure according to claim 8, wherein a ratio between the plurality of dummy structures and the plurality of polyproline helix structures in each of the detecting regions is 3:1 or greater than 3:1.

10. A production method of a detection structure, comprising:
providing a substrate, the substrate has a plurality of detecting regions;
coating a coating structure on the substrate;
providing a plurality of polyproline helix structures, wherein each of the polyproline helix structures is composed of a repeating helical arrangement of a plurality of proline monomers aligned in a first direction, a plurality of proline monomers aligned in a second direction, and a plurality of proline monomers aligned in a third direction;
modifying at least one of the plurality of proline monomers aligned in the first direction with at least one connecting structure, so that the plurality of proline monomers aligned in the first direction is connected to the coating structure in each of the detecting regions through the connecting structure;
connecting the plurality of proline monomers aligned in the second direction with at least two ligands, so that the two ligands on each of the polyproline helix structures has a fixed distance that can be adjusted; and
providing a plurality of dummy structures and connecting the plurality of dummy structures within the coating structure in each of the detecting regions, so that the plurality of dummy structures and the plurality of polyproline helix structures are adjacent to one another.

11. The production method of the detection structure according to claim 10, wherein the coating structure is a fluorous coating structure, and the plurality of proline monomers aligned in the first direction is modified with perfluorinated alkyl moiety of the connecting structure, so that the plurality of proline monomers aligned in the first direction is connected to the coating structure in each of the detecting regions through the perfluorinated alkyl moiety.

12. The production method of the detection structure according to claim 11, wherein the plurality of proline monomers aligned in the first direction is modified with perfluorinated alkyl moiety of the connecting structure, and the perfluorinated alkyl moiety is a perfluorinated alkyl moiety having more than three carbons.

13. The production method of the detection structure according to claim 11, wherein the plurality of proline monomers aligned in the first direction is modified with at least two chains of the perfluorinated alkyl moiety of the connecting structure.

14. The production method of the detection structure according to claim 11, wherein the plurality of proline monomers aligned in the second direction is connected to the at least two ligands through covalent bonding.

15. The production method of the detection structure according to claim 10, wherein the plurality of dummy structures is connected to the coating structure by a non-covalent interaction through perfluorinated alkyl moiety.

16. The production method of the detection structure according to claim 10, wherein a ratio between the plurality of dummy structures and the plurality of polyproline helix structures in each of the detecting regions is 3:1 or greater than 3:1.

17. The production method of the detection structure according to claim 10, wherein the fixed distance is 0.9±0.1 nm, 1.8±0.1 nm, 2.7±0.1 nm, 3.6 nm±0.1 nm or 4.5 nm±0.1 nm.
